(19) United States
(12) Patent Application Publication
(10) Pub. No.: US 2006/0094901 A1
(43) Pub. Date: May 4, 2006

(54) Process for the production of carboxylic acids

(76) Inventors: Keith Whiston, (US); Graham Howard Jones, (US)

Correspondence Address: INVISTA NORTH AMERICA S.A.R.L., THREE LITTLE FALLS CENTRE/1052, 2801 CENTERVILLE ROAD, WILMINGTON, DE, 19808, US

(21) Appl. No.: 10/979,285

(22) Filed: Nov. 2, 2004

(51) Int. Cl. C07C 51/16

(52) U.S. Cl. 562/545

(57) ABSTRACT

A process for the production of a carboxylic acid or its ester by catalytic liquid phase oxidation of a corresponding precursor in acetic acid as solvent, said process comprising: (i) forming a reaction medium comprising acetic acid, oxidation catalyst, precursor and oxidant; (ii) optionally recycling methyl acetate produced from the acetic acid as a by-product back to the reaction medium; (iii) introducing additional methyl acetate and/or methanol into the reaction medium, said additional methyl acetate and/or methanol being additional to any recovered methyl acetate recycled back to the reaction medium.

### CROSS REFERENCE TO RELATED APPLICATION

This application claims priority benefit of U.S. Provisional Patent Application Ser. No. 60/378,311 filed 6 May 2002.

### FIELD OF THE INVENTION

This invention relates to a process for producing carboxylic acids, particularly terephthalic acid.

### DESCRIPTION OF THE PRIOR ART

Terephthalic acid is an important intermediate in the production of polyesters used, for instance, in the manufacture of fibres, bottles and films. Oxidation of para-xylene with molecular oxygen in a lower (e.g. C₂-C₆) aliphatic monocarboxylic acid, usually acetic acid, as solvent in the presence of a catalyst system containing one or more heavy metals such as cobalt or manganese and a promoter such as bromine is well known as the standard method for the preparation of terephthalic acid. Acetic acid is particularly useful as the solvent since it is relatively resistant to oxidation in comparison with other solvents and increases the activity of the catalytic pathway. Although this method is favoured for the commercial production of terephthalic acid, there remains a problem in that loss of the acetic acid solvent takes place during the reaction. This loss of carbon from the process increases the cost of operation. The acetic acid loss occurs as a result of combustion of acetic acid to form carbon oxides (CO and CO₂) and as a result of the formation of methyl acetate and/or methanol as by-product(s). The formation of methyl acetate and/or methanol from acetic acid can account for about 20-30% of the total carbon loss.

The combustion of acetic acid has previously been investigated and various solutions for controlling the combustion have been proposed, for example, specific reaction conditions or a specific catalyst system.

The other principal mechanism for loss of solvent, i.e., the formation of methyl acetate, was addressed in U.S. Pat. No. 4,239,493 and U.S. Pat. No. 4,560,793. These documents disclose a process for the production of terephthalic acid in acetic acid solvent, wherein the vapour effluent evolved from the oxidation reaction containing the methylacetate by-product is passed through a condenser which recovers a portion of this by-product as a condensate. The remaining methylacetate in the off-gas from the condenser is recovered by scrubbing the off-gas with acetic acid and the recovered methylacetate is recirculated to the reaction. The resulting increased concentration of methylacetate in the reaction mother liquor has the effect of suppressing the formation of methylacetate from acetic acid, thereby reducing the amount of acetic acid solvent lost. Roffia P. et al. (Ind. Eng. Chem Res. 1988, 27, 765-770) reports a study on the interdependence of methyl acetate production and process variables and the advantages obtained from recycling methyl acetate to the oxidation reaction.

### DESCRIPTION OF THE INVENTION

It is an object of this invention to provide a process for minimising non-productive carbon loss, in particular loss of acetic acid solvent, in the manufacture of a carboxylic acid by catalytic liquid phase oxidation of a corresponding precursor, particularly the manufacture of terephthalic acid by the oxidation of p-xylene.

Accordingly, the present invention provides a process for the production of a carboxylic acid or its ester by catalytic liquid phase oxidation of a corresponding precursor in acetic acid as solvent, said process comprising (i) forming a reaction medium comprising acetic acid, oxidation catalyst, precursor and oxidant; (ii) optionally recycling methyl acetate produced from the acetic acid as a by-product back to the reaction medium; (iii) introducing additional methyl acetate and/or methanol into the reaction medium, said additional methyl acetate and/or methanol being additional to any recovered methyl acetate recycled back to the reaction medium.

The optional recycling of methyl acetate produced as a by-product back to the reaction medium may be achieved by passing the vapour effluent containing the methyl acetate by-product from the reaction medium through a condenser. A portion of said methyl acetate in said vapour is recovered as a condensate from the condenser. At least part and preferably substantially all of the remaining methyl acetate is recovered from the off-gas of the condenser by scrubbing the off-gas with acetic acid. The methyl acetate thereby recovered is re-circulated to the reaction medium.

The methanol or additional methyl acetate may be introduced directly into the reaction medium or may be introduced, for instance, into the acetic acid feed stream prior to or concurrently with entry of the acetic acid feed stream into the reaction medium.

The process of the present invention is advantageous in that it reduces the amount of non-productive carbon loss in the reaction. There is a reduced formation rate of methyl acetate in the oxidation reaction medium and no increase in the formation of carbon oxides (CO and CO₂). The process is of beneficial application in two cases.

Firstly in artificially augmenting the concentration of methyl acetate and/or methanol in the reaction medium in an oxidation plant already practising a high degree of methyl acetate recycle, i.e. in a plant practicing a recycle as per the process of U.S. Pat. No. 4,329,493. An additional benefit in reducing acetic loss is derived in this way.

Secondly, in oxidation plants in which some or all of the methyl acetate by-product is lost from the reaction and fuel value benefit is derived from the increase in methyl acetate in the vapour effluent or vent gas. Thus, this application is suitable for plants where an off-gas abatement facility is employed to which support fuel is routinely added in order to maintain the unit's operating temperature. Such processes are generally used commercially only where the economics of acetic acid and methyl acetate purchase allow it, i.e. where the cost of the support fuel and/or methyl acetate/methanol is low enough to counter balance the acetic acid loss, or for compliance with local environmental regulations to minimise emission of carbon oxides and, for instance, methyl bromide. A plant of this type utilises catalytic combustion, for instance as disclosed in WO-A-96/39595.

The amount of additional methyl acetate and/or methanol is no more than 4 mole %, preferably no more than 3 mole %, and more preferably no more than 2 mole % of the solvent feed. Preferably, the molar concentration is at least 0.5%. Preferably, the molar concentration is 1-2%. If the concentration of added methyl acetate or methanol is too high, detrimental side reactions such as the build-up of formic acid begin to dominate and an increase in the carbon oxides in the vent gas per mole of para-xylene feedstock is observed.

As noted above, the concept of recycling methyl acetate in a terephthalic acid manufacturing process is well-known and practiced commercially and it is in such plants where the present invention is envisaged to be of most benefit. In the known process, methyl acetate is recycled through the oxidation reactor in order to save acetic acid solvent by inhibiting formation of methyl acetate therefrom, as described in U.S. Pat. No. 4,239,493 and U.S. Pat. No. 4,560,793 and by Roffia et al. In contrast, the present invention introduces additional, non-recycled methyl acetate or methanol into the reactor, and accrues a corresponding additional benefit in reducing acetic loss. Unexpectedly, the process of the present invention achieves this additional benefit without increase in the undesirable formation of carbon oxides as by-products. Thus, there is no increase in the vent concentrations of CO or CO₂ per mole of para-xylene feedstock when the concentration of methyl acetate or methanol in the reactor is increased by the introduction of additional methyl acetate or methanol. This result is particularly surprising in view of the prior disclosure by Roffia et al who teach that the methanol produced as a result of the methyl acetate recycle is decomposed to CO and CO₂. It is therefore unexpected from this prior disclosure that addition of fresh methyl acetate or methanol does not increase the formation of carbon oxides.

In one aspect, the present invention therefore provides an unexpected advantage in decreasing non-productive carbon loss, in particular loss of acetic acid solvent, in a manufacturing process for carboxylic acids which practices methyl acetate recycle. Thus, there is an economic benefit in increasing the methyl acetate or methanol concentration in the feed to the reactor which results in a reduction of methyl acetate formation from acetic acid without increase in CO or CO₂ formation per mole of p-xylene feedstock.

In a second aspect, the present invention provides, in plants operating an off-gas abatement facility, economic benefit as a result of the fuel value obtainable from the unexpected absolute increase in volatile organic compounds (VOCs), i.e. methyl acetate and/or methanol, in the vapour effluent or off-gas from the reactor. The absolute increase in VOCs in the vapour effluent or off-gas is derived from the additional methyl acetate or methanol introduced into the system according to the present invention. According to the conventional wisdom, for instance as described by Roffia et al, it would have been expected that any additional methanol and/or methyl acetate would have decomposed to CO and CO₂ before reaching the catalytic combustion unit. The present invention teaches that additional methanol and/or methyl acetate introduced into the system is not lost via the formation of carbon oxides but unexpectedly remains available for use as fuel in an off-gas abatement facility, provided that the additional methyl acetate and/or methanol is added in the amounts described herein.

In either aspect, the teaching of the prior art would not have justified this process modification or suggested the resulting economic benefits.

The process of the present invention may also be used in a plant operating both partial methyl acetate recycle and off-gas abatement. Thus, in plants operating at less than 100% recycle there will be some methyl acetate/methanol in the off-gas available for use as fuel in the catalytic combustion unit.

The modification of the manufacturing process described herein is applicable to both new plant designs and retro fitting to existing plants.

The invention is described herein primarily in relation to terephthalic acid. However, it will be appreciated that the following is also applicable to the production of carboxylic acids or their esters generally, particularly phthalic acids or their esters, by catalytic liquid phase oxidation of a corresponding precursor.

In general terms, the catalytic liquid phase oxidation of p-xylene to produce terephathalic acid comprises feeding acetic acid, oxidant, para-xylene and catalyst into an oxidation reactor that is maintained at a temperature in the range from 150° C. to 250° C., preferably 175° C. to 225° C., and a pressure in the range from 100 to 5000 kPa, preferably 1000 to 3000 kPa. The feed acetic acid: para-xylene ratio is typically less than 5:1.

The oxidation catalyst is preferably a homogeneous catalyst, i.e. it is soluble in the reaction medium comprising solvent and the aromatic carboxylic acid precursor(s). The catalyst typically comprises one or more heavy metal compounds, e.g. cobalt and/or manganese compounds, and may optionally include an oxidation promoter. For instance, the catalyst may take any of the forms that have been used in the liquid phase oxidation of aromatic carboxylic acid precursors in aliphatic carboxylic acid solvent, e.g. bromides, bromoalkanoates or alkanoates (usually C₁-C₄ alkanoates such as acetates) of cobalt and/or manganese. Compounds of other heavy metals such as vanadium, chromium, iron, molybdenum, a lanthanide such as cerium, zirconium, hafnium, and/or nickel may be used instead of or in addition to cobalt and/or manganese. Advantageously, the catalyst system will include cobalt bromide (CoBr₂) and/or manganese bromide (MnBr₂). The oxidation promoter where employed may be in the form of elemental bromine, ionic bromide (e.g. HBr, NaBr, KBr, NH₄Br) and/or organic bromide (e.g. bromobenzenes, benzyl-bromide, mono- and di-bromoacetic acid, bromoacetyl bromide, tetrabromoethane, ethylene-di-bromide, etc.). Alternatively the oxidation promoter may comprise a ketone, such as methylethyl ketone, or aldehyde, such as acetaldehyde.

The oxidant in the process of the invention is preferably molecular oxygen, e.g. air or oxygen-enriched air. Instead of molecular oxygen, the oxidant may comprise atomic oxygen derived from a compound, e.g. a liquid phase compound at room temperature, containing one or more oxygen atoms per molecule. One such compound is hydrogen peroxide, which acts as a source of oxygen by reaction or decomposition.

Oxidant (preferably molecular oxygen) is added in amounts in excess of the stoichiometric requirements for full conversion of the paraxylene to terephthalic acid, to minimise formation of undesirable by-products, such as color formers. Immediately upon entering the reactor, the paraxylene is thoroughly mixed with the oxygenated solvent to initiate the reaction. The oxidation reaction is exothermic, and heat may be removed by allowing the acetic acid solvent to vaporise. The corresponding vapour is condensed and most of the condensate is refluxed to the reactor, with some condensate being withdrawn to control reactor water concentration (two moles of water are formed per mole of paraxylene reacted). The residence time is typically 30 minutes to 2 hours, depending on the process.

The effluent, i.e. reaction product, from the oxidation reactor is a slurry of crude terephthalic acid (TA) crystals which are recovered from the slurry by filtration, washed, dried and conveyed to storage. They are thereafter fed to a separate purification step or directly to a polymerization process. The main impurity in the crude TA is 4-carboxybenzaldehyde (4-CBA), which is incompletely oxidized paraxylene, although p-tolualdehyde and p-toluic acid can also be present along with undesirable color formers.

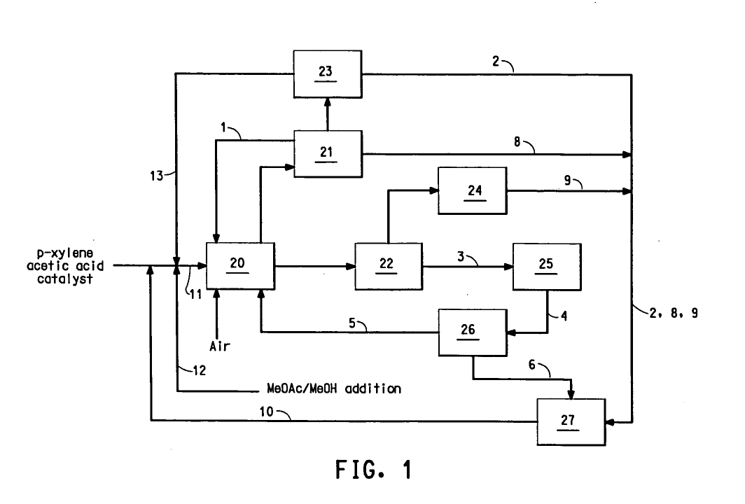

The invention in one of its embodiments is illustrated by FIG. 1 showing a p-xylene oxidation process in which full methyl acetate recycle is being practiced. Methyl acetate is conventionally recycled to the oxidation reactor in a number of ways. Methyl acetate in the acetic acid reflux from the primary reactor (20) is directly returned to the reactor (20) from condensor (21) via the reflux return line (1). Vent gasses from the primary reactor and any downstream crystallisation vessels (22) are scrubbed in scrubbers (23 (high pressure) and 24) with acetic acid and water. The extraction solvent from this process is then sent to the solvent recovery process for recovery of acetic acid solvent and methyl acetate (2), or recycled directly to the reactor feed (13).

Methyl acetate present in the product stream (3) from the primary reactor is contained mainly in the process mother liquor. The mother liquor containing methyl acetate (4) is conventionally separated from the product terephthalic acid in filtration unit (25). The filtrate or process mother liquor (4) is then split at stream splitter (26) into two parts: a direct recycle stream (5) and a purge stream (6). The direct recycle stream (5) is then returned, with the contained methyl acetate, directly to the oxidation reactor (20) via the reactor feed. The mother liquor purge stream (6) is sent to a distillation system (27) for the recovery of acetic acid solvent.

The condensate withdrawal (8) normally taken from the overheads condenser system (21) on the primary reactor (20) is also sent to the solvent recovery system (27) together with some or all of the solvent used for scrubbing methyl acetate from the primary reactor vent (2) and from the crystalliser vents (9). A methyl acetate rich stream (10) is then conventionally produced within the solvent recovery system (27) from these combined sources of recovered methyl acetate. This recovered methyl acetate is then recycled to the primary reactor (20) by mixing with directly recycled mother liquor and fresh solvent feed as stream (11). In feeding the oxidation reactor (20), stream (11) can conventionally be combined with streams (5) or (13) and some or all of stream (1) before the primary reactor.

The invention described above constitutes the addition of methyl acetate or methanol (12) into the reactor feed (11) or the mother liquor recycle stream (5) or any combination of these with stream (1), the reflux return.

The invention is further illustrated by the following examples. It will be appreciated that the examples are for illustrative purposes only and are not intended to limit the invention described above. Modification of detail may be made without departing from the scope of the invention.

### EXPERIMENTAL

The following examples illustrate how the invention can be demonstrated on a continuous small-scale oxidation facility. It is not practical to recycle methyl acetate directly on a pilot oxidation vessel. This is due to the difficulty of recovering and recycling both the process mother liquor and the volatile components in the vent completely. Therefore it is necessary to simulate methyl acetate recycle on such a unit as described below.

The percentage of methyl acetate (MeOAc) recycle achieved in a process is defined by:
% Methyl acetate recycle=(MeOAc in the reactor feed moles per mole p-Xylene/MeOAc leaving the reactor moles per mole p-Xylene)×100

In order to simulate methyl acetate recycle on a pilot oxidation unit, one gradually increases the concentration of methyl acetate in the reactor feed by substituting part of the acetic acid solvent with methyl acetate. At the same time the concentration of methyl acetate leaving the unit in the mother liquor, via the condensate withdrawal and through the reactor vents is monitored. As the concentration of methyl acetate in the feed increases, the percentage of simulated recycle also increases until the concentration in the feed exactly balances the measured concentration leaving the oxidation. This state is 100% methyl acetate recycle and simulates the effect of a manufacturing plant recycling all the internally generated methyl acetate until a steady state is reached.

Example 1 below illustrates the invention by showing results from an experiment carried out at above 100% methyl acetate recycle, giving the vent carbon oxides loss and total carbon loss measured for the experiment. Comparative example 1 gives results from an identical experiment where the degree of simulated MeOAc recycle was 89%. Example 1 clearly shows a benefit over comparative example 1 in reducing total carbon loss from the reaction. Example 2 shows results from an identical experiment to example 1 but with no simulated methyl acetate recycle. This further illustrates the benefit of operation at greater than 100% methyl acetate recycle and the advantage of adding some further MeOAc to the reactor solvent feed.

Example 3 and comparative example 2 illustrate that the invention operates using a different catalyst system and under different temperature conditions by comparing an experiment at 101% simulated methyl acetate recycle with another at 83% methyl acetate recycle as with example 1 and comparative example 1.

Example 4 illustrated how the invention can be practised using methanol (MeOH) rather than MeOAc as the additive.

Results are presented in Tables 1 and 2.

Example 5, comparative example 3 and example 6 show that when higher concentrations of MeOAc and MeOH were included in the reactor feed, catalyst activity deteriorated significantly (as shown by the very significant 4-CBA increase) and the selectivity deteriorated (no coincident reduction in burn was observed).

In comparing results from a p-xylene oxidation reaction for improvements in selectivity or variations in catalyst activity it is important to compare like with like.

### Example 1

A zirconium pressure vessel of 5 litre capacity equipped with a stirrer, a reflux condenser with condensate withdrawal facility, an air inlet, a heater, a feed inlet line and a slurry discharge line was charged with 3000 g of an acetic acid solution containing water 8%, methyl acetate 2.3%, cobalt 200 ppm, manganese 400 ppm, sodium 100 ppm and bromide 800 ppm. Cobalt, sodium and manganese were added as their acetate salts. Bromide was added as hydrogen bromide.

The vessel was then heated to 213° C. and 19 barg pressure and maintained under these conditions with agitation. An acetic acid feed stream of the following composition was continuously added to the pressure vessel at a rate of 3100 g/hr: p-xylene 18%, water 5.5%, cobalt 120 ppm, manganese 240 ppm, sodium 60 ppm, bromide 490 ppm, methyl acetate 2.28%. Air was also added to the vessel at such a rate as to maintain the reactor vent oxygen concentration at 3.5%. Condensate was withdrawn continuously from the pressure vessel at the rate of 1000 g/hr. Product slurry was continuously discharged from the autoclave into a pressure let down vessel before sampling.

After 6 hours of continuous operation, the carbon loss from the reaction was determined. This was achieved by measuring the concentration of by-product carbon oxides, methyl acetate, methanol, methyl bromide, methane and methyl formate in the vent gases discharged from both the oxidation autoclave and let down vessel, and the methyl acetate and methanol in the withdrawn condensate liquid from the reaction and the discharged product slurry. The loss was then calculated as g-atom carbon per mole of p-xylene in the reactor feed. The total concentration of methyl acetate in the reactor feed as g-atom carbon per mole of p-xylene in the feed was then subtracted from this total to give the net carbon loss from the reaction. The percentage of methyl acetate recycle was also calculated from the reactor feed concentration and the measured concentration in the vents, mother liquor and withdrawn condensate samples.

The product terephthalic acid contained 4000 ppm of 4-carboxybenzaldehyde. The extent of simulated methyl acetate recycle was 103%. The total loss of carbon oxides from the reaction was 0.25 moles/mole p-xylene in the feed. The net total carbon loss was 0.25 g-atom carbon/mole p-xylene fed.

### Comparative Example 1

An experiment as example 1 was carried out but with 1.5% methyl acetate in the reactor charge and 1.5% methyl acetate in the reactor feed. The extent of simulated methyl acetate recycle was 89%. The product terephthalic acid contained 3500 ppm of 4-carboxybenzaldehyde. The total loss of carbon oxides from the reaction was 0.26 moles/mole p-xylene in the feed. The net total carbon loss was 0.30 g-atom carbon/mol p-xylene fed.

### Example 2

An experiment as example 1 was carried out but with no methyl acetate in either the feed or in the reactor charge. Thus, there was no methyl acetate recycle practiced in this experiment. The product terephthalic acid contained 3500 ppm of 4-carboxybenzaldehyde. The total loss of carbon oxides from the reaction was 0.28 moles/mole p-xylene in the feed. The net total carbon loss was 0.36 g-atom carbon/mol p-xylene fed.

### Example 3

An experiment as in example 1 was carried out but at a reactor temperature of 180° C. and air was added to the vessel at such a rate as to maintain the reactor vent oxygen concentration at 5%. The initial reactor charge consisted of 3000 g of an acetic solution containing 8% water, 2.5% methyl acetate, 1400 ppm cobalt, 470 ppm manganese, 100 ppm sodium and 1870 ppm bromide. A continuous acetic acid feed of the following composition was fed to the autoclave at a rate of 2300 g/hr: p-xylene 18%, water 4.1%, methyl acetate 2%, cobalt 840 ppm, manganese 280 ppm, sodium 60 ppm, bromide 1120 ppm. Condensate was withdrawn continuously from the reactor at a rate of 730 g/hr.

The terephthalic acid produced had a 4-carboxybenzaldehyde concentration of 3400 ppm. The extent of simulated methyl acetate recycle was 101%. The total loss of carbon oxides from the reaction was 0.19 moles/mole p-xylene in the feed. The net total carbon loss was 0.19 g-atom carbon/mol p-xylene fed.

### Comparative Example 2

An experiment identical to example 3 was carried out but with 1% methyl acetate in the autoclave feed during this period. The extent of simulated methyl acetate recycle was 83%. The terephthalic acid produced had a 4-carboxybenzaldehyde concentration of 2800 ppm. The total loss of carbon oxides from the reaction was 0.19 moles/mole p-xylene in the feed. The net total carbon loss was 0.22 g-atom carbon/mol p-xylene fed.

### Example 4

An experiment as in example 1 was carried out but at a reactor temperature of 185° C. and air was added to the vessel at such a rate as to maintain the reactor vent oxygen concentration at 5%. The initial reactor charge consisted of 3000 g of an acetic solution containing 8% water, 1% methanol, 900 ppm cobalt, 300 ppm manganese, 100 ppm sodium and 1200 ppm bromide. A continuous acetic acid feed of the following composition was fed to the autoclave at a rate of 2300 g/hr; p-xylene 18%, water 4.1%, methanol 0.85%, cobalt 540 ppm, manganese 180 ppm, sodium 60 ppm, bromide 720 ppm. Condensate was withdrawn continuously from the reactor at a rate of 730 g/hr. The methanol addition is the molar equivalent to a concentration of 2% methyl acetate ion the reactor feed. The terephthalic acid produced had a 4-carboxybenzaldehyde concentration of 4300 ppm. The total loss of carbon oxides from the reaction was 0.20 moles/mole p-xylene in the feed. The net total carbon loss was 0.09 g-atom carbon/mol p-xylene fed.

Results for the above examples are given in Table 1.

### Example 5

An experiment as in example 1 was carried out but air was added to the vessel at such a rate as to maintain the reactor vent oxygen concentration at 1.9%. The initial reactor charge consisted of 3000 g of an acetic solution containing 15% water, 6.3% methyl acetate, 210 ppm cobalt, 560 ppm manganese, 400 ppm sodium and 870 ppm bromide. A continuous acetic acid feed of the following composition was fed to the autoclave at a rate of 1850 g/hr: p-xylene 24%, water 4.1%, methyl acetate 6.3%, cobalt 210 ppm, manganese 560 ppm, sodium 400 ppm, bromide 870 ppm. No condensate was withdrawn from the reactor.

The terephthalic acid produced had a 4-carboxybenzaldehyde concentration of 3800 ppm. The total loss of carbon oxides from the reaction was 0.39 moles/mole p-xylene in the feed after 6 hours.

### Comparative Example 3

An experiment was carried out as in example 5, but no methyl acetate was present in the autoclave charge or reactor feed. The total loss of carbon oxides from the reaction was 0.39 moles/mole p-xylene in the feed after 6 hours, but the terephthalic acid produced had a 4-carboxybenzaldehyde concentration of 2900 ppm.

### Example 6

An experiment was carried out as in example 5, but 2.74% methanol was present in the autoclave charge and reactor feed instead of the methyl acetate. The total loss of carbon oxides from the reaction was 0.40 moles/mole p-xylene in the feed after 6 hours, but the terephthalic acid produced had a 4-carboxybenzaldehyde concentration of 3900 ppm.

The results for examples 5 and 6 and comparative example 3 are given in table 2.

TABLE 1

| | Product | MeOAc | MeOAc in | MeOAc out | CO + CO₂ | MeOAc | Total carbon by- | Methyl Acetate |
|---|---|---|---|---|---|---|---|---|
| | 4CBA | in feed | mole/mole | Mole/mole | Mole/mole | net out | production g-atom | Recycle |
| | % | ppm | pX | pX | pX | mole/mole pX | C/mole pX | % |
| Example 1 | 0.41 | 22830 | 0.193 | 0.187 | 0.254 | −0.006 | 0.246 | 102.0 |
| Comparative | 0.36 | 11290 | 0.095 | 0.107 | 0.258 | 0.012 | 0.299 | 89.0 |
| Example 1 | | | | | | | | |
| Example 2 | 0.35 | 0 | 0 | 0.026 | 0.275 | 0.026 | 0.356 | 0 |
| Example 3 | 0.28 | 20000 | 0.143 | 0.142 | 0.188 | −0.001 | 0.186 | 101.0 |
| Comparative | 0.34 | 10000 | 0.072 | 0.087 | 0.189 | 0.015 | 0.223 | 83.0 |
| Example 2 | | | | | | | | |
| Example 4 | 0.43 | 20118 | N/A | 0.102 | 0.205 | N/A | 0.085 | N/A |
| | | (MeOH) | | | | | | |

TABLE 2

| | Product 4CBA | MeOAc in feed | CO + CO₂ |
|---|---|---|---|
| | % | ppm | mole/mole pX |
| Example 5 | 0.38 | 63300 | 0.39 |
| Comparative | 0.28 | 0 | 0.39 |
| Example 5 | | | |
| Example 6 | 0.38 | 27400 | 0.4 |
| | | (MeOH) | |

1. A process for the production of a carboxylic acid or its ester by catalytic liquid phase oxidation of a corresponding precursor in acetic acid as solvent, said process comprising:
(i) forming a reaction medium comprising acetic acid, oxidation catalyst, precursor and oxidant;
(ii) optionally recycling methyl acetate produced from the acetic acid as a by-product back to the reaction medium;
(iii) introducing additional methyl acetate and/or methanol into the reaction medium, said additional methyl acetate and/or methanol being additional to any recovered methyl acetate recycled back to the reaction medium.

2. A process according to claim 1 wherein the carboxylic acid is terephthalic acid and said precursor is p-xylene.

3. A process according to claim 1 wherein the methyl acetate produced from the acetic acid as a by-product is recycled back to the reaction medium.

4. A process according to claim 1 wherein said recycling of methyl acetate produced as a by-product back to the reaction medium comprises passing the vapour effluent containing the methyl acetate by-product from the reaction medium through a condenser.

5. A process according to claim 4 wherein at least a portion of said methyl acetate in said vapour is recovered as a condensate from the condenser.

6. A process according to claim 4 wherein at least a portion of the methyl acetate in the off-gas of the condenser is recovered by scrubbing the off-gas with acetic acid and/or water.

7. A process according to claim 5 wherein at least a portion of the methyl acetate in the off-gas of the condenser is recovered by scrubbing the off-gas with acetic acid and/or water.

8. A process according to claim 6 wherein at least a portion of said methyl acetate and acetic acid resulting from scrubbing the off-gas of the condenser is recycled directly back to the reaction medium.

9. A process according to claim 7 wherein at least a portion of said methyl acetate and acetic acid resulting from scrubbing the off-gas of the condenser is recycled directly back to the reaction medium.

10. A process according to claim 6 wherein at least a portion of said methyl acetate and acetic acid resulting from scrubbing the off-gas of the condenser is passed to distillation for recovery of acetic acid and methyl acetate and recycle to the reaction medium.

11. A process according to claim 7 wherein at least a portion of said methyl acetate and acetic acid resulting from scrubbing the off-gas of the condenser is passed to distillation for recovery of acetic acid and methyl acetate and recycle to the reaction medium.

12. A process according to claim 8 wherein at least a portion of said methyl acetate and acetic acid resulting from scrubbing the off-gas of the condenser is passed to distillation for recovery of acetic acid and methyl acetate and recycle to the reaction medium.

13. A process according to claim 9 wherein at least a portion of said methyl acetate and acetic acid resulting from scrubbing the off-gas of the condenser is passed to distillation for recovery of acetic acid and methyl acetate and recycle to the reaction medium.

14. A process according to claim 1 wherein said process for the production of carboxylic acid comprises crystallising the reaction product in one or more crystallisation vessel(s) and wherein said recycling of methyl acetate comprises scrubbing the off-gas of the crystallisation vessel(s) with acetic acid and/or water.

15. A process according to claim 1 wherein said process for the production of carboxylic acid comprises crystallising the reaction product and recovering the carboxylic acid crystals by filtration and wherein said recycling of methyl acetate comprises passing at least a portion of the filtrate to distillation for recovery of acetic acid and methyl acetate and recycle to the reaction medium.

16. A process according to claim 15 wherein at least a portion of the filtrate is recycled directly to the reaction medium.

17. A process according to claim 1 wherein said additional methyl acetate and/or methanol is no more than 4 mole % of the solvent feed.

18. A process according to claim 1 wherein said additional methyl acetate and/or methanol is no more than 3 mole % of the solvent feed.

19. A process according to claim 1 wherein said additional methyl acetate and/or methanol is no more than 2 mole % of the solvent feed.
